(19) United States
(12) Patent Application Publication
(10) Pub. No.: US 2010/0198146 A1
(43) Pub. Date: Aug. 5, 2010

(54) APPARATUS AND METHOD FOR DELIVERING BIOLOGICALLY-ACTIVE SUBSTANCES OR MICRO-MEDICAL DEVICES TO A TARGET

(76) Inventor: Gopalan Jagadeesh, Bangalore (IN)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 12/480,514

(22) Filed: Jun. 8, 2009

(30) Foreign Application Priority Data
Feb. 5, 2009 (IN) 260/CHE/2009

(51) Int. Cl. A61M 5/303

(52) U.S. Cl. 604/69

(57) ABSTRACT

A solid or liquid material that includes a biologically-active substance, or a micro-medical device is acoustically coupled to a propulsion surface of a diaphragm. A blast-receiving surface of the diaphragm is acoustically coupled to an explosion chamber in which an explosive material is disposed. An ignition system ignites the explosive material in the explosion chamber to create a blast wave. The diaphragm transfers momentum from the blast wave to the solid or liquid material or the micro-medical device sufficient to propel the solid or liquid material or the micro-medical device across a standoff distance to reach a target.

### BACKGROUND

As scientists continue to discover the genetic causes of many diseases, the need for safe and effective gene therapy increases. Gene therapy may be a solution to major diseases such as cancer, cardiovascular disease, and inherited metabolic disorders, among other diseases and disorders. See Kodama, et al., Cytoplasmic Molecular Delivery with Shock Waves: Importance of Impulse, 79 BIOPHYSICAL JOURNAL 1821, 1821 (October 2000). A device that is able to safely and effectively deliver biologically-active particles could be useful in cancer treatment, HIV treatment, and other treatments involving genetic therapies.

Localized drug delivery is advantageous to traditional drug delivery because the biologically-active particles are delivered directly to the treatment site, avoiding side effects such as bleeding or stroke. See Shangguan, et al., Drug Delivery with Microsecond Laser Pulses into Gelatin, 35 APPLIED OPTICS 3347, 3347 (Jul. 1, 1996). Particles can be delivered into living cells by accelerating these particles to speeds in which they can penetrate the cells without destroying them. See Klein, et al., High-velocity Microprojectiles for Delivering Nucleic Acids into Living Cells, 327 NATURE 70, 70 (May 1987).

Inexpensive, safe, and effective gene therapy and drug delivery is needed in developing countries to treat diseases such as HIV. Non-invasive devices are desirable because they allow for liquid drug suspensions and drug formulations to be delivered into the patient without the need for any carrier or physical contact. Ideally, developing countries in need of these devices could manufacture the devices themselves using low cost consumables. In addition, these devices must be portable, so that they can be easily used and transported.

Accordingly, there continues to be a need to miniaturize devices used to deliver biologically-active substances to a target.

### SUMMARY

In a first aspect, an illustrative embodiment provides an apparatus for delivering a biologically-active substance to a target. The apparatus includes but is not limited to an explosion chamber, an explosive material disposed in the explosion chamber, an ignition system for igniting the explosive material in the explosion chamber to create a blast wave, and a diaphragm with a blast-receiving surface acoustically coupled the explosion chamber and a propulsion surface opposite the blast-receiving surface. The apparatus also includes but is not limited to a solid or liquid material that includes but is not limited to the biologically-active substance, wherein the solid or liquid material is acoustically coupled to the propulsion surface such that the diaphragm is able to transfer momentum from the blast wave to the solid or liquid material sufficient to propel the solid or liquid material across a standoff distance to reach the target.

In other illustrative embodiments of this apparatus, the biologically-active substance includes but is not limited to a gene.

In other illustrative embodiments of this apparatus, the solid or liquid material includes but is not limited to a liquid in contact with the propulsion surface of the diaphragm.

In other illustrative embodiments, the apparatus further includes but is not limited to a container holding the liquid, the container including but not limited to a bottom wall opposite the propulsion surface of the diaphragm. The bottom wall includes but is not limited to an orifice, the orifice holding the liquid in the container by surface tension when the liquid is quiescent and allows the liquid to flow out of said container when the liquid receives the momentum from the blast wave.

In other illustrative embodiments, the apparatus propels the solid or liquid material into the target so as to reach a penetration depth in the target.

In other illustrative embodiments of this apparatus, the penetration depth includes but is not limited to between about 100 μm and about 800 μm.

In other illustrative embodiments of this apparatus, the diaphragm includes but is not limited to a metal foil having a thickness between about 100 μm and about 200 μm.

In a second aspect, an illustrative embodiment provides method for propelling particles to a target. The method includes but is not limited to depositing the particles on a propulsion surface of a diaphragm and positioning the diaphragm such that the propulsion surface faces the target and is separated from the target by a standoff distance. The method also includes but is not limited to positioning a tube, the tube having a first open end and a second open end, such that the first open end is acoustically coupled to a blast-receiving surface of the diaphragm opposite the propulsion surface and the tube having an explosive material disposed therein. The method further includes but is not limited to igniting the explosive material in the tube to create a blast wave, wherein the diaphragm transfers momentum from the blast wave to the particles sufficient to propel the particles across the standoff distance to reach the target.

Other illustrative embodiments of this method include but are not limited to particles are coated with a biologically-active substance.

In other illustrative embodiments of this method, the target includes but is not limited to a living organism.

In other illustrative embodiments of this method, depositing the particles on a propulsion surface of a diaphragm includes but is not limited to applying a liquid suspension of the particles to the propulsion surface of the diaphragm.

In other illustrative embodiments of this method, igniting the explosive material in the tube to create a blast wave includes but is not limited to applying electrical energy from a power supply to the explosive material.

In other illustrative embodiments of the method, the standoff distance is between about 1 mm and about 8 mm.

In other illustrative embodiments of this method, the particles penetrate into the target to reach a penetration depth of between about 100 μm and about 800 μm.

In another aspect, an illustrative embodiment provides an apparatus for delivering a micro-medical device to a target. The apparatus includes but is not limited to an explosion chamber, an explosive material disposed in the explosion chamber, an ignition system for igniting the explosive material in the explosion chamber to create a blast wave, and a diaphragm having a blast-receiving surface acoustically coupled to the explosion chamber and a propulsion surface opposite the blast-receiving surface, wherein the micro-medical device is acoustically coupled to the propulsion surface such that the diaphragm is able to transfer momentum from the blast wave to the micro-medical device sufficient to propel the micro-medical device across a standoff distance to reach the target.

In other illustrative embodiments of the apparatus, the standoff distance is between about 1 mm and about 8 mm.

In other illustrative embodiments of this apparatus, the apparatus is able to propel the micro-medical device into the target so as to reach a penetration depth in the target.

In other illustrative embodiments of the apparatus, the diaphragm includes but is not limited to a metal foil having a thickness between about 100 μm and about 200 μm.

In other illustrative embodiments, the apparatus is integrated with an endoscope.

In other illustrative embodiments, the apparatus is integrated with an intravenous delivery system.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1 is a schematic diagram of a delivery apparatus, in accordance with an illustrative embodiment.

FIG. 2 is a perspective view of an ignition system, in accordance with an illustrative embodiment.

FIG. 3 is a schematic diagram of a particle propulsion apparatus, in accordance with an illustrative embodiment.

FIG. 4 is a schematic diagram of the ignition system of FIG. 2, in accordance with an illustrative embodiment.

FIG. 5 is a graph of penetration depths of particles in relation to agarose strength, in accordance with an illustrative embodiment.

FIG. 6 is a digital image of a 0.6% agarose gel target with penetrated tungsten particles, in accordance with an illustrative embodiment.

FIG. 7 is a digital image of particle scatter at a target surface, in accordance with an illustrative embodiment.

FIG. 8 is a digital image of tungsten particles delivered into Arachis hypogea, in accordance with an illustrative embodiment.

FIG. 9 is a digital image of tungsten particles delivered into ground tissue of a potato tuber, in accordance with an illustrative embodiment.

FIG. 10 is a schematic diagram of a liquid propulsion apparatus, in accordance with an illustrative embodiment

FIG. 11 is a digital image of a 5% agarose gel target with penetrated liquid jet, in accordance with an illustrative embodiment.

FIG. 12 is a digital image of a 1% agarose gel target showing the penetration depths of SAE oils of different grades, in accordance with an illustrative embodiment

FIG. 13 is a digital image of a liquid jet delivered into Morus alba, in accordance with an illustrative embodiment.

FIG. 14 is a digital image of a liquid jet delivered into Piper nigrum, in accordance with an illustrative embodiment.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

### 1. OVERVIEW

To deliver a biologically-active substance or a micro-medical device to a target, an explosion chamber, explosive material, and an ignition system may be arranged to create a blast wave. The explosive material may be disposed in the explosion chamber and the ignition system may be used to ignite the explosive material and create the blast wave. The explosion container may comprise a polymer tube. The apparatus may include a diaphragm, which may have a blast-receiving surface acoustically coupled to the explosion chamber and a propulsion surface opposite the blast-receiving surface. One example of a diaphragm may be a metal foil having a thickness between about 100 μm and about 200 μm.

The biologically active substance may be included in a solid or liquid material. The biologically-active substance may comprise a gene or a drug. The solid or liquid material or the micro-medical device may be acoustically coupled to the propulsion surface of the diaphragm such that the diaphragm is able to transfer momentum from the blast wave to the solid or liquid material or the micro-medical device sufficient to propel the material or device across a standoff distance to reach the target. The target may be a living organism. The solid or liquid material may comprise solid particles disposed on the propulsion surface of the diaphragm. The solid particles may also be coated with a biologically-active substance. The solid or liquid material may also comprise a liquid in contact with the propulsion surface of the diaphragm.

The apparatus may also comprise a container holding the liquid and the container may have a bottom wall arranged opposite of the propulsion surface of the diaphragm. The bottom wall may have an orifice, which holds the liquid by surface tension when the liquid is quiescent and allows the liquid to flow out of the container when the liquid receives momentum from the blast wave.

The apparatus may be configured such that the standoff distance is between about 1 mm and about 8 mm. The apparatus also may be configured so that it is able to propel the solid or liquid particle or micro-medical device into the target to reach a penetration depth in the target, for example, a penetration depth between about 100 μm and about 800 μm.

### 2. HANDHELD DEVICE

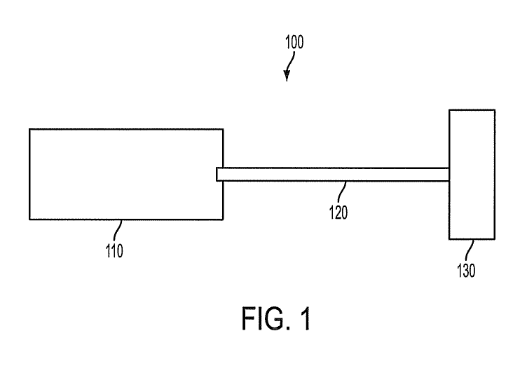

FIG. 1 schematically illustrates a handheld device 100 that includes an ignition system 110, an explosion chamber 120, and a cell transformation apparatus 130. Device 100 may be used to deliver biologically-active particles or a micro-medical device into a target, as described in more detail below.

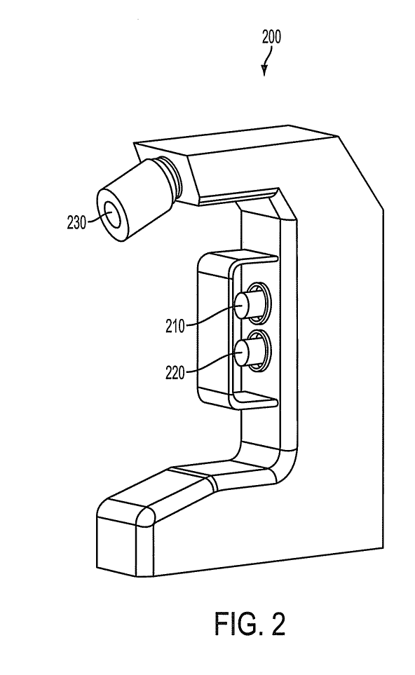

FIG. 2 is a perspective view of an illustrative ignition system 200 that may be used in device 100. Ignition system 200 may be dimensioned to be conveniently held by hand. Ignition system 200 includes a charging switch 210, a firing switch 220, and an electrode 230, with electrode 230 connected to explosion chamber 120. Ignition system 200 may be used to ignite explosive material in explosion chamber 120 and thereby create a blast wave that can be used to deliver biologically-active particles or a micro-medical device into a target. In operation, the user may activate charging switch 210 to charge ignition system 200 and then activate firing switch 220 to ignite the explosive material. Ignition system 200 may be arranged in various configurations in addition to that illustrated in FIG. 2.

### 3. PARTICLE PROPULSION APPARATUS

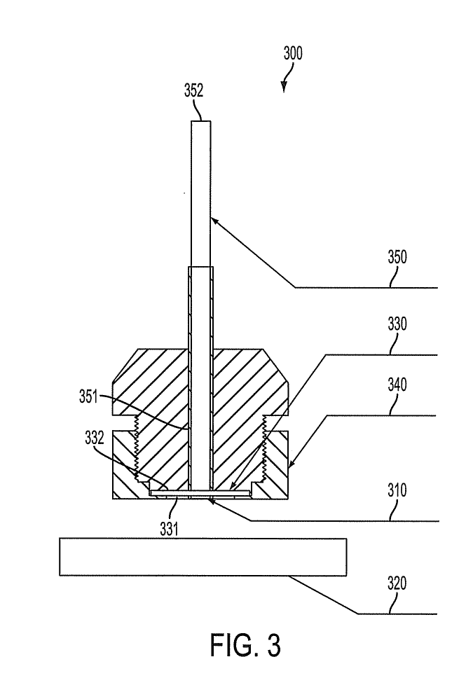

FIG. 3 illustrates a particle propulsion apparatus 300 that may be used to propel one or more solid particles 310 to a target 320. Particles 310 could be micro-particles (particles that are on the order of one micron in diameter). Alternatively, particles 310 could be larger, or smaller (e.g., nano-particles). For example, particles 310 may be tungsten or gold. Particles 310 may also be one or more micro-medical devices, such as cameras, for example. These cameras could be used to view the intestinal tract. Particles 310 may also be coated with a biologically-active substance. For example, particles 310 may be a polymer coated with a gene or a drug. Target 320 may be made from a variety of materials. For example, target 320 may be an agarose gel or a living organism.

Apparatus 300 may include a diaphragm 330 and a diaphragm holder 340. Diaphragm 330 has a propulsion surface 331 on one side and a blast-receiving surface 332 on an opposite side. Diaphragm 330 may be a metal foil, such as aluminum, copper, brass, or silver. In illustrative embodiments, the thickness of diaphragm 330 is between about 100 μm and 200 μm and the diameter of diaphragm 330 is about 12 mm. Diaphragm holder 340 may include multiple pieces connected together in order to hold diaphragm 330 firmly in place.

Particles 310 may be deposited on propulsion surface 331 of diagram 330 by applying a liquid suspension of particles 310. In one example, 10 mg of tungsten (0.7 micron) particles are suspended in 1 ml of ethanol (C₂H₅OH) and a volume of 0.5 μL of the suspension is deposited on a metal diaphragm. The ethanol evaporates leaving behind approximately 150,000 tungsten particles on the diaphragm. Depending on the desired distribution and number of particles, the concentration (ppm level) of the particles and the volume to be deposited may be varied.

Diaphragm 330 can be positioned such that propulsion surface 331 faces target 320 and is separated from target 320 by a standoff distance. In illustrative embodiments, the standoff distance is between about 1 mm and 8 mm.

Apparatus 300 also may include a tube 350 that functions as an explosion chamber. Tube 350 has a first open end 351 and a second open end 352, such that first open end 351 is acoustically coupled to blast-receiving surface 332 of diaphragm 330. In a representative embodiment, tube 350 may have a 1 mm inside diameter and a wall thickness of 1 mm. Tube 350 may be made from a variety of materials. For example, tube 350 may be a polymer. In a representative embodiment, tube 350 is a three-layer polymer, in which the inner layer is ionomer and the middle and outer layers are polyethylene. Tube 350 may also be made out of stainless steel.

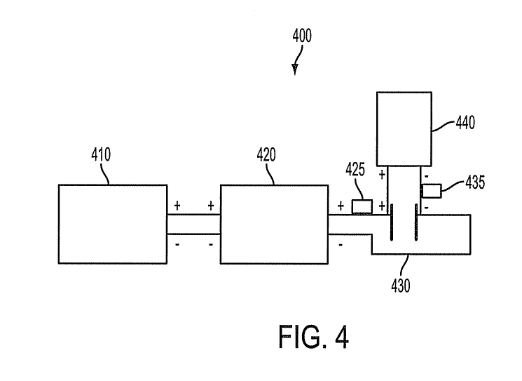

Tube 350 may contain explosive material, which can be ignited to create a blast wave. The explosive material may be coated on the inner wall of tube 350. In an example embodiment, the explosive material is a mixture of HMX and aluminum with a particle size of 20 microns, wherein the aluminum is applied at 2 mg/m length and the HMX is applied at 16 mg/m length. The explosive material may be ignited by applying electrical energy from an ignition system, such as shown in FIG. 4. Tube 350 may also be electrically coupled to the ignition system by inserting at least one electrode into second open end 351. Diaphragm 330 then transfers momentum from the blast wave to particles 310 sufficient to propel the particles across the standoff distance. In illustrative embodiments, particles 310 reach a penetration depth of between about 100 μm and about 800 μm.

Apparatus 300 may also be integrated with an endoscope for targeted drug delivery or integrated with an intravenous delivery system for intravenous therapy.

FIG. 4 illustrates an ignition system 400 that may be used to ignite the explosive material. Ignition system 400 may include a power supply 410, a voltage converter 420, a capacitor 430, and an electrode 440. In illustrative embodiments, there is a spark gap in electrode 440, which may be in the range of about 0.5 mm to about 1 mm. Power supply 410 may also include a charging switch 425, located between voltage convertor 420 and capacitor 430, and a firing switch 435, located between capacitor 430 and electrode 440. In an illustrative embodiment, power supply 410 is a 9V alkaline battery and capacitor 430 is a 0.2 μF capacitor. Voltage converter 420 converts the 9V to 2500V. When the user activates charging switch 425, capacitor 430 is charged up to 2500V. When the user activates the firing switch 435, capacitor 430 discharges through electrode 440 to create a spark. The spark ignites the explosive material to create a blast wave. The blast wave travels the length of tube 350. In an illustrative embodiment, the blast wave travels at a rate of about 2000 m/s. The blast wave deforms diaphragm 330. During the process of deformation, diaphragm 330 transfers the momentum from the blast to particles 310 such that the particles are propelled across the standoff distance and penetrate target 320.

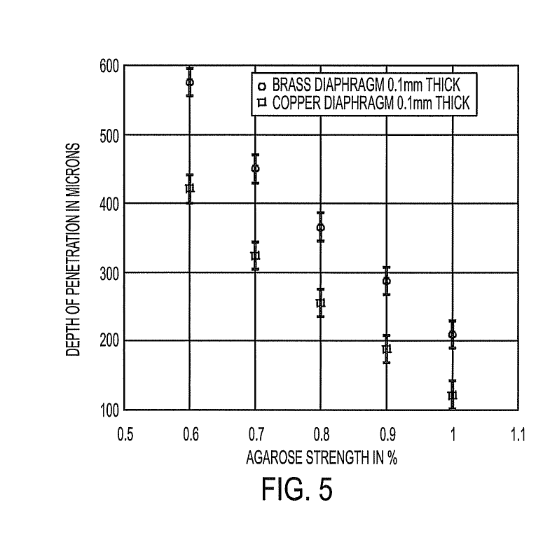

Agarose gel targets may have strengths varying from 0.6% to 1.0%. The percentage of agarose is determined by weight ratio of agarose powder to water. FIG. 5 shows penetration depths varying from 210 μm (in 1% agarose gel) to 560 μm (in 0.6% agarose gel) when using a 0.1 mm thick brass diaphragm at a standoff distance of 4 mm and shows penetration depths varying from 120 μm (in 1% agarose gel) to 420 μm (in 0.6% agarose gel) when using a 0.1 mm thick copper diaphragm at a standoff distance of 4 mm. Table 1 below shows the data from FIG. 5 in tabular format.

TABLE 1

| | Brass | |
|---|---|---|
| Agarose | Diaphragm (0.1 mm | Copper Diaphragm (0.1 mm |
| Concentration % | thick) Average Value | thick) Average Value |
| 0.6 | 576 | 421 |
| 0.7 | 450 | 324 |
| 0.8 | 366 | 256 |
| 0.9 | 288 | 188 |
| 1 | 210 | 122 |

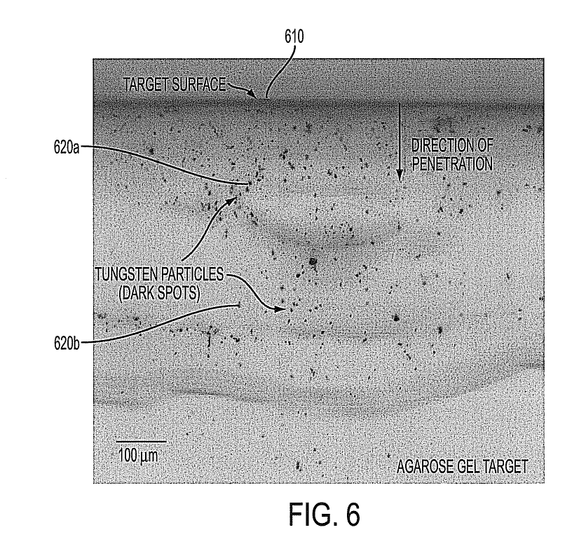

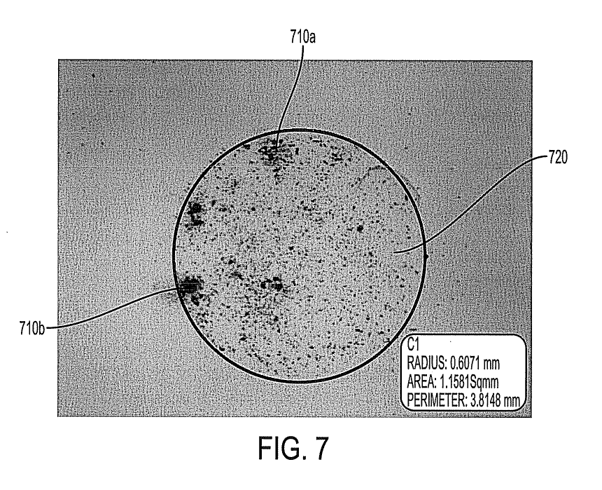

FIGS. 6 and 7 illustrate microscopic investigations of the distribution of the particles and penetration topology. FIG. 6 shows an agarose gel target 610 of 0.6% strength in which tungsten particles 620a and 620b have penetrated from a standoff distance of 4 mm. FIG. 7 shows particle scatter 710a and 710b at a surface of a target 720.

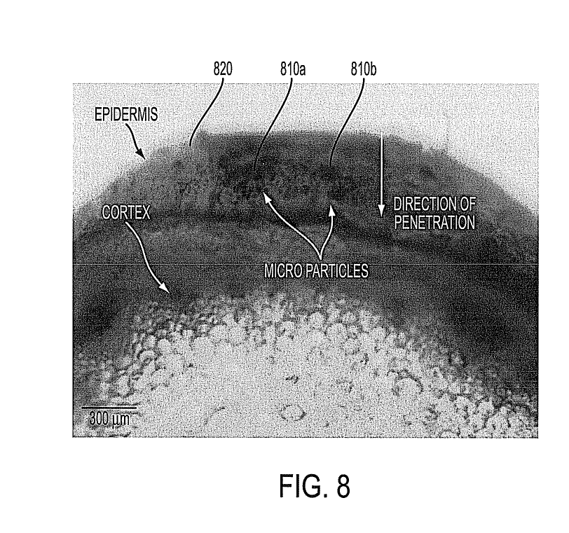

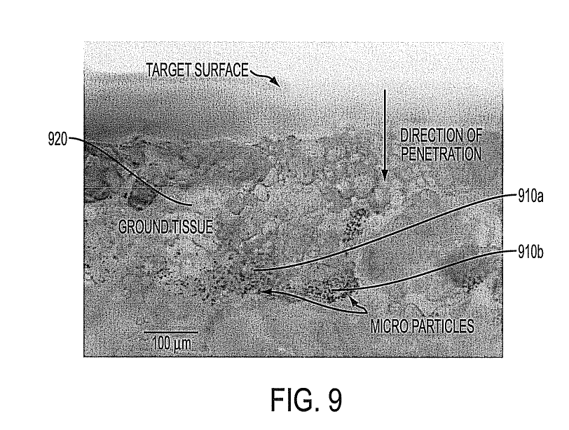

Targets can also include cells from living organisms. FIG. 8 shows tungsten particles 810a and 810b delivered into a target 820 of a living plant cell of Arachis hypogea at a standoff distance of 4 mm. FIG. 9 shows tungsten particles 910a and 910b delivered into a target 920 of the ground tissue of a potato tuber at a standoff distance of 4 mm.

Illustrative embodiments of the particle propulsion apparatus have been described above. It is to be understood, however, that a particle propulsion apparatus could be constructed and/or used in other ways.

### 4. LIQUID JET PROPULSION APPARATUS

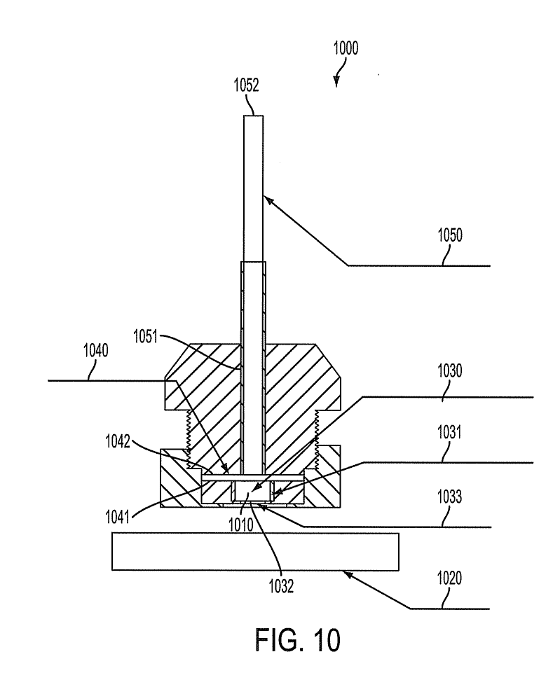

FIG. 10 illustrates a liquid jet propulsion apparatus 1000 that may be used to propel a liquid 1010 to a target 1020. Liquid 1010 may be, for example, water-based or oil-based and may include dissolved or suspended materials. Liquid 1010 may also include a biologically-active substance, such as a gene or a drug, which may be either dissolved in the liquid or coated on particles (e.g., gold or tungsten micro-particles). Target 1020 may be made from a variety of materials. For example, target 1020 may be an agarose gel or a living organism.

Apparatus 1000 may include a container 1030, which has a container lining 1031, a bottom wall 1032 and an orifice 1033 located in bottom wall 1032. Container 1030 may be cylindrical in shape. Container 1030 may be made from a bio-inert material, such as MACOR® machinable glass-ceramic, or 316L stainless steel. The volume of container 1030 may vary. For example, the volume may be 20 μL, 36 μL, or 57 μL, where the depth of the container is 3 mm and the diameter is varied. In some embodiments, container lining 1031 may be a biologically inert material such as Teflon® polymer. Orifice 1033 may be of such a size that it holds the liquid in the container by surface tension. For example, orifice 1033 may have a diameter of 300 μm. Container 1030 may be positioned such that orifice 1033 faces target 1020 and is separated from the target by a standoff distance. In illustrative embodiments, the standoff distance is between about 1 mm and about 8 mm.

Apparatus 1000 also may include a diaphragm 1040 that has a propulsion surface 1041 on one side and a blast-receiving surface 1042 on an opposite side. Diaphragm 1040 may be positioned such that its propulsion surface 1041 contacts liquid 1010 in container 1030. Diaphragm 1040 may be a metal foil, such as aluminum, copper, brass, or silver. In illustrative embodiments, the thickness of diaphragm 1040 is between about 100 μm and 200 μm. Diaphragm 1040 may be similar to diaphragm 330 in FIG. 3. Apparatus 1000 also may include a tube 1050 that has a first open end 1051 and a second open end 1052, such that first open end 1051 is acoustically coupled to blast-receiving surface 1042 of diaphragm 1040. Tube 1050 may be similar to tube 350 in FIG. 3. Tube 1050 contains explosive material, which is ignited to create a blast wave. The explosive material may be similar to the explosive material in FIG. 3. The explosive material may be ignited by applying electrical energy from an ignition system, such as ignition system 400 shown in FIG. 4. Tube 1050 may also be electrically coupled to the power supply by inserting at least one electrode into second open end 1052. Diaphragm 1040 then transfers momentum from the blast wave to the liquid sufficient to propel liquid 1010 across the standoff distance. In illustrative embodiments, liquid 1010 reaches a penetration depth of between about 100 μm and about 800 μm.

Apparatus 1000 may also be integrated with an endoscope for targeted drug delivery or integrated with an intravenous delivery system for intravenous therapy.

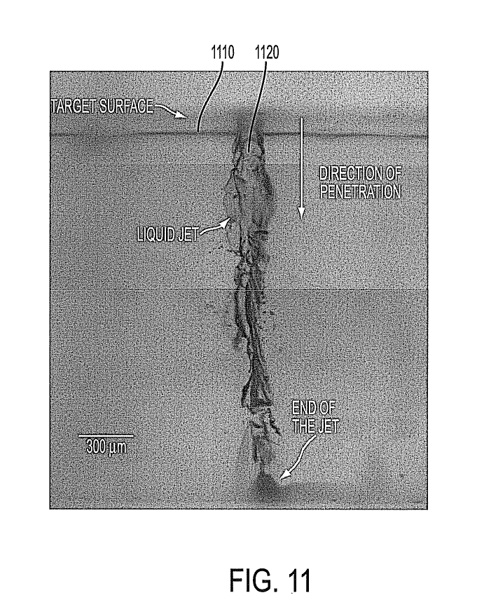

In illustrative embodiments, liquid jets can be delivered into agarose gel targets of strengths varying from 1% to 5%, and penetration depths of up to 2000 μm can be achieved. FIG. 11 shows an agarose target 1110 of 5% strength and a standoff distance of 1 mm with a penetrated liquid jet 1120. In this example, water containing a dye is the fluid used in the liquid jet.

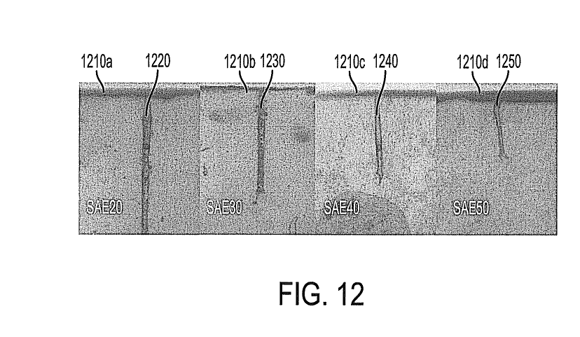

In other embodiments, the liquid may be a high-viscosity fluid. For example, FIG. 12 shows 1% agarose targets 1210a-d at standoff distances of 1 mm with penetration depths of oils of different SAE grades (SAE20 oil 1220, SAE30 oil 1230, SAE40 oil 1240, and SAE50 oil 1250), resulting in penetration depths varying from 470 μm to 1640 μm. Table 2 below shows the relationship between the kinematic viscosity and density of the various oils and the depth of penetration achieved.

TABLE 2

| | Kinematic | | Depth of |
|---|---|---|---|
| | Viscosity | Density | penetration |
| Type of Oil | (mm²/s) | (kg/m³) | (μm) |
| SAE10 | 115 | 870 | 10125 |
| SAE20 | 200 | 885 | 8500 |
| SAE30 | 350 | 890 | 5500 |
| SAE40 | 900 | 900 | 4300 |
| SAE50 | 950 | 902 | 2900 |

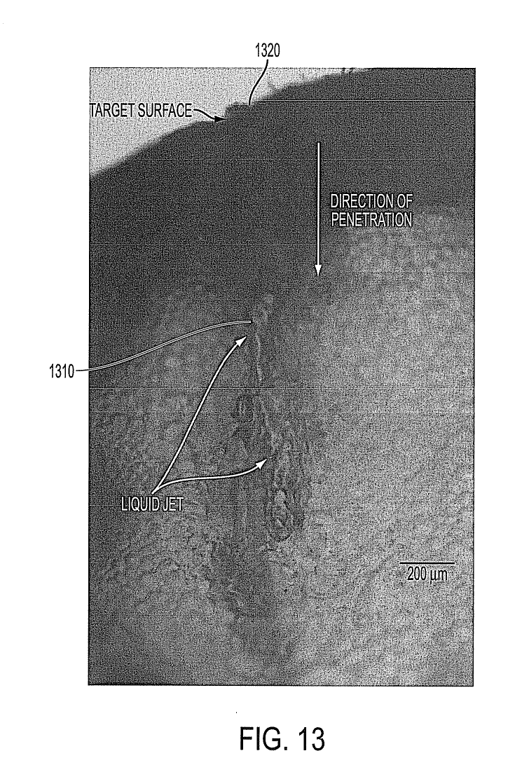

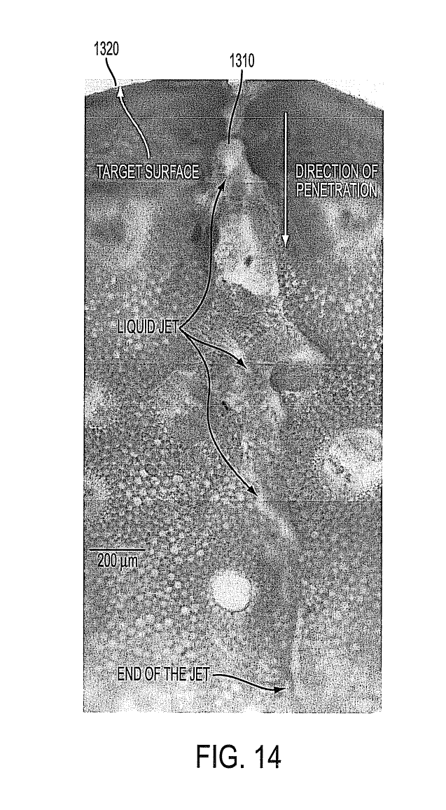

In other example embodiments, liquid jets are delivered into living plant cells. FIG. 13 shows a liquid jet 1310 delivered into a target 1320 of Morus alba, commonly known as white mulberry, which is a host plant for silk worms FIG. 14 shows a liquid jet 1410 delivered into a target 1420 of Piper nigrum, commonly known as black pepper. Penetration depths of more than 2000 μm may be reached.

Illustrative embodiments of the liquid propulsion apparatus have been described above. It is to be understood, however, that a liquid propulsion apparatus could be constructed and/or used in other ways.

### 5. ILLUSTRATIVE APPLICATIONS

By using apparatuses as shown and described here, genetic material or drugs can be delivered into living organisms, including humans, without the need for needles or syringes, reducing the risk of transmission of blood-born diseases. These apparatuses may be useful for the treatment of cancer, HIV, and other diseases. These apparatuses may also be used to genetically modify living plant cells. Because these devices may be handheld devices, they can be easy to use and transport. These devices may also be integrated with an endoscope based device for targeted drug delivery. In addition, these devices may be used where conventional gene guns are used, such as in biotechnology industries and laboratories.

### 6. CONCLUSION

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions, or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. An apparatus for delivering a biologically-active substance to a target, comprising:
an explosion chamber;
an explosive material disposed in said explosion chamber;
an ignition system for igniting said explosive material in said explosion chamber to create a blast wave;
a diaphragm having a blast-receiving surface acoustically coupled to said explosion chamber and a propulsion surface opposite said blast-receiving surface; and
a solid or liquid material that includes said biologically-active substance, wherein said solid or liquid material is acoustically coupled to said propulsion surface such that said diaphragm is able to transfer momentum from said blast wave to said solid or liquid material sufficient to propel said solid or liquid material across a standoff distance to reach said target.

2. The apparatus of claim 1, wherein said biologically-active substance comprises a gene.

3. The apparatus of claim 1, wherein said solid or liquid material comprises a liquid in contact with said propulsion surface of said diaphragm.

4. The apparatus of claim 1, further comprising:
a container holding a liquid, said container having a bottom wall opposite said propulsion surface of said diaphragm, said bottom wall having an orifice therein, said orifice having a size that holds said liquid in said container by surface tension when said liquid is quiescent and allows said liquid to flow out of said container when said liquid receives said momentum from said blast wave.

5. The apparatus of claim 1, wherein said apparatus is able to propel said solid or liquid material into said target so as to reach a penetration depth in said target.

6. The apparatus of claim 5, wherein said penetration depth is between about 100 μm and about 800 μm.

7. The apparatus of claim 1, wherein said diaphragm comprises a metal foil having a thickness between about 100 μm and about 200 μm.

8. A method for propelling particles to a target, comprising:
depositing said particles on a propulsion surface of a diaphragm;
positioning said diaphragm such that said propulsion surface faces said target and is separated from said target by a standoff distance;
positioning a tube, said tube having a first open end and a second open end, such that said first open end is acoustically coupled to a blast-receiving surface of said diaphragm opposite said propulsion surface, said tube having an explosive material disposed therein; and
igniting said explosive material in said tube to create a blast wave, wherein said diaphragm transfers momentum from said blast wave to said particles sufficient to propel said particles across said standoff distance to reach said target.

9. The method of claim 8, wherein said particles are coated with a biologically-active substance.

10. The method of claim 8, wherein said target is a living organism.

11. The method of claim 8, wherein depositing said particles on a propulsion surface of a diaphragm comprises:
applying a liquid suspension of said particles to said propulsion surface of said diaphragm.

12. The method of claim 8, wherein igniting said explosive material in said tube to create a blast wave comprises:
applying electrical energy from a power supply to said explosive material in said tube.

13. The method of claim 8, wherein said standoff distance is between about 1 mm and about 8 mm.

14. The method of claim 8, wherein said particles penetrate into said target to reach a penetration depth of between about 100 μm and about 800 μm.

15. An apparatus for delivering a micro-medical device to a target, comprising:
an explosion chamber;
an explosive material disposed in said explosion chamber;
an ignition system for igniting said explosive material in said explosion chamber to create a blast wave; and
a diaphragm having a blast-receiving surface acoustically coupled to said explosion chamber and a propulsion surface opposite said blast-receiving surface, wherein said micro-medical device is acoustically coupled to said propulsion surface such that said diaphragm is able to transfer momentum from said blast wave to said micro-medical device sufficient to propel said micro-medical device across a standoff distance to reach said target.

16. The apparatus of claim 15, wherein said standoff distance is between about 1 mm and about 8 mm.

17. The apparatus of claim 15, wherein said apparatus is able to propel said micro-medical device into said target so as to reach a penetration depth in said target.

18. The apparatus of claim 15, wherein said diaphragm comprises a metal foil having a thickness between about 100 μm and about 200 μm.

19. The apparatus of claim 15, wherein said apparatus is integrated with an endoscope.

20. The apparatus of claim 15, wherein said apparatus is integrated with an intravenous delivery system.
